(19) United States
(12) Patent Application Publication
(10) Pub. No.: US 2026/0157793 A1
(43) Pub. Date: Jun. 11, 2026

(54) ADJUSTMENT OF PHYSIOLOGIC FUNCTION SUPPLEMENTATION CONTROL

(71) Applicant: Cilag GmbH International, Zug (CH)

(72) Inventors: Frederick E. Shelton, IV, Hillsboro, OH (US); Jason L. Harris, Lebanon, OH (US); Matthew David Cowperthwait, Cincinnati, OH (US); Shane R. Adams, Lebanon, OH (US); Nicholas James Ross, Franklin, OH (US); Thomas Allen Stoughton, Cincinnati, OH (US)

(21) Appl. No.: 18/971,933

(22) Filed: Dec. 6, 2024

(51) Int. Cl. A61B 18/14; A61B 17/00; A61B 18/00; G16H 40/63

(52) U.S. Cl. CPC A61B 18/1492; G16H 40/63; A61B 2017/00199; A61B 2017/00212; A61B 2018/00351; A61B 2018/00577; A61B 2018/00648; A61B 2018/00678; A61B 2018/00898; A61B 2018/00994

(57) ABSTRACT

A surgical device may manage physiologic supplementation during procedures. The device may assess the patient's status and adjust supplementation in real time based on a control signals. A feedback mechanism may monitor an effect, and the device may introduce a controlled change. The device may apply a localized intervention, such as temperature management, to an area of a body, and use a data source to maintain a desired condition throughout the procedure.

### CROSS-REFERENCE TO RELATED APPLICATIONS

This application is related to the following, filed contemporaneously, the contents of each of which are incorporated by reference herein:
U.S. patent application Ser. No. 18/971,590 entitled PROGRESSIVE ADVANCEMENT OF AUTOMATED LEVEL BASED ON LEARNED COMPLIMENTARY ASSISTANCE
U.S. patent application Ser. No. 18/971,596 entitled ADJUSTING AUTOMATED COOPERATIVE OPERATIONS BASED ON SITUATIONALLY DERIVED CONSTRAINTS,
U.S. patent application Ser. No. 18/971,606 entitled ASSISTANCE ADVANCEMENT MULTI-SYSTEM INTERACTION,
U.S. patent application Ser. No. 18/971,609 entitled MONITORING AND IDENTIFYING SURGEON CONTROL AND SUGGESTING A TASK THAT MAY BE DONE AUTONOMOUSLY,
U.S. patent application Ser. No. 18/971,861 entitled CONTROL OF INFORMATION FLOW, PRIORITIZATION AND MANIFESTATION OF DATA ASSOCIATED WITH AN ACTIVE HCP INTERACTION SPACE,
U.S. patent application Ser. No. 18/971,888 entitled ADAPTIVE RETRACTION FORCE CONTROL, and
U.S. patent application Ser. No. 18/971,908 entitled ADJUSTMENT OR DISPLAY OF OPTIONS OF POSITIONAL OR ORIENTATION IMPLICATIONS ON SURGICAL TOOL USAGE.

The contents of each of the following are incorporated by reference herein:
U.S. patent application Ser. No. 18/810,323 entitled METHOD FOR MULTI-SYSTEM INTERACTION, filed on Aug. 20, 2024;
U.S. patent application Ser. No. 18/960,006 entitled METHOD FOR SMART SURGICAL SYSTEMS filed on Nov. 26, 2024; and
U.S. patent application Ser. No. 18/954,186 entitled METHOD FOR MULTI-SYSTEM INTERACTION, filed on Nov. 20, 2024.

### BACKGROUND

Surgical procedures are typically performed in surgical operating theaters or rooms in a healthcare facility such as, for example, a hospital. Various surgical devices and systems are utilized in performance of a surgical procedure. In the digital and information age, medical systems and facilities are often slower to implement systems or procedures utilizing newer and improved technologies due to patient safety and a general desire for maintaining traditional practices.

### SUMMARY

Examples described herein may be associated with a surgical device that manages physiologic function supplementation during medical procedures. The device may include a processor that receives indications of a patient's physiologic functions, such as temperature, heart rate, or other relevant data. The device may evaluate whether the functions are within predefined ranges and generate control signals based on the information to adjust physiologic function supplementation. The system may operate through continuous monitoring and adaptive adjustments to maintain the desired physiologic state throughout the procedure.

The surgical device may integrate safety features to enable adjustments to remain within operational limits, preventing overcorrection or unsafe interventions. The device may assess the magnitude and rate of adjustments to keep them within acceptable ranges. The device may include feedback mechanisms that monitor the outcomes of the adjustments, providing a real-time check to ensure that the adjustments are effectively managing the physiologic functions. Such configuration may allow the device to adapt to changes in the patient's condition during the procedure.

In examples, the device may introduce (e.g., small) perturbations to evaluate the patient's physiologic response. The perturbations may aid in distinguishing between physiologic changes and potential sensor errors. The device's ability to make adaptive changes based on feedback may enable physiologic function supplementation to progress toward an outcome.

The device may enable interventions (e.g., complex interventions), such as localized temperature management, which may be applied to specific areas of the patient's body. Such localized temperature management may enable precise control over physiologic functions while avoiding unintended effects on other parts of the body. The feedback and control system may process data from multiple sensors and systems and manage physiologic function supplementation based on real-time data and responses from the patient.

### BRIEF DESCRIPTION OF THE DRAWINGS

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 shows an example surgical system that may include a surgical instrument.

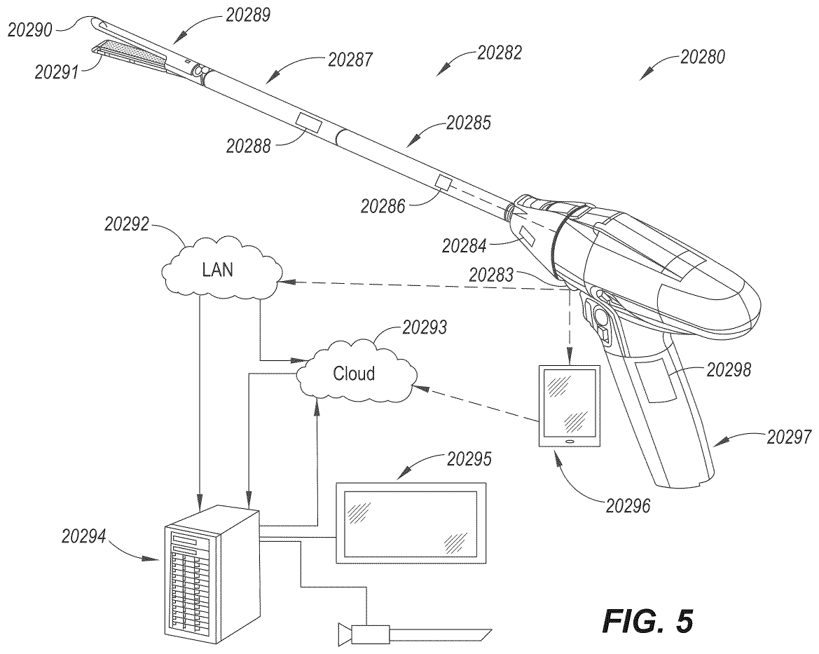

FIG. 6 illustrates an example of a patient undergoing a procedure utilizing a warming system (e.g., a bear hugger warming system) and a local cooling device to manage physiologic function supplementation through thermal regulation.

FIG. 7 illustrates a flow diagram for an example of monitoring and adjusting physiologic function supplementation.

FIG. 8 illustrates an example machine learning diagram used to process user data and health outcomes for adaptive intervention and feedback control.

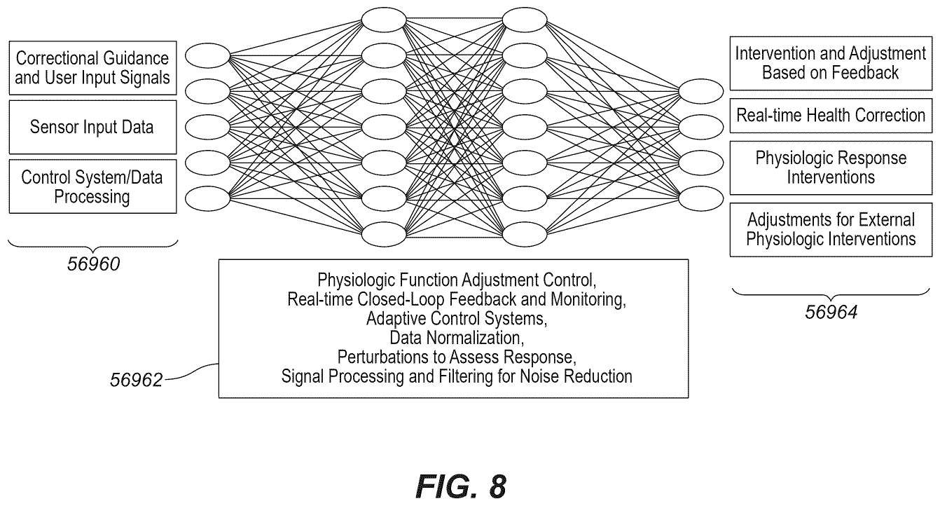

### DETAILED DESCRIPTION

A more detailed understanding may be had from the following description, given by way of example in conjunction with the accompanying drawings.

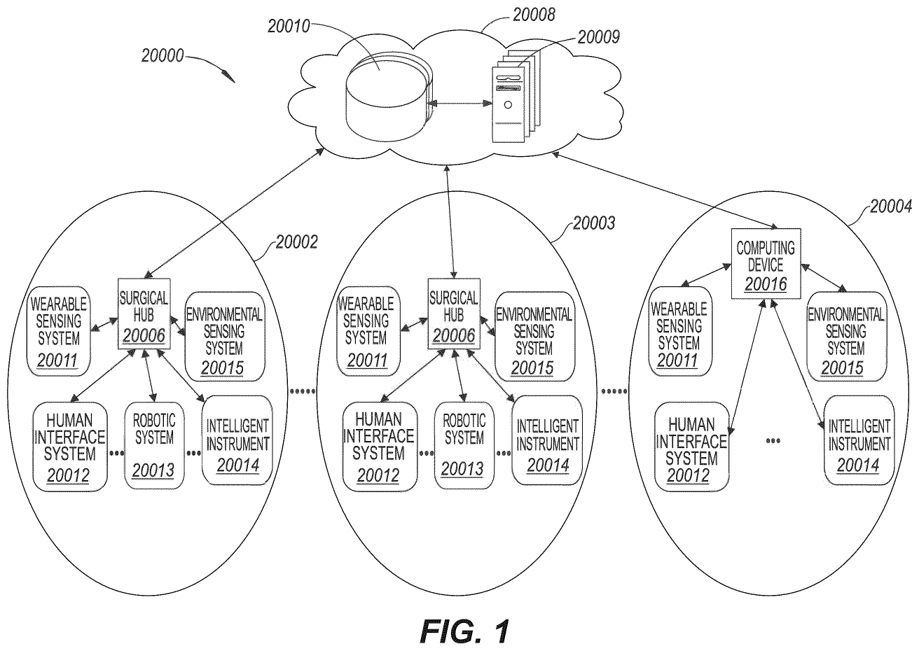

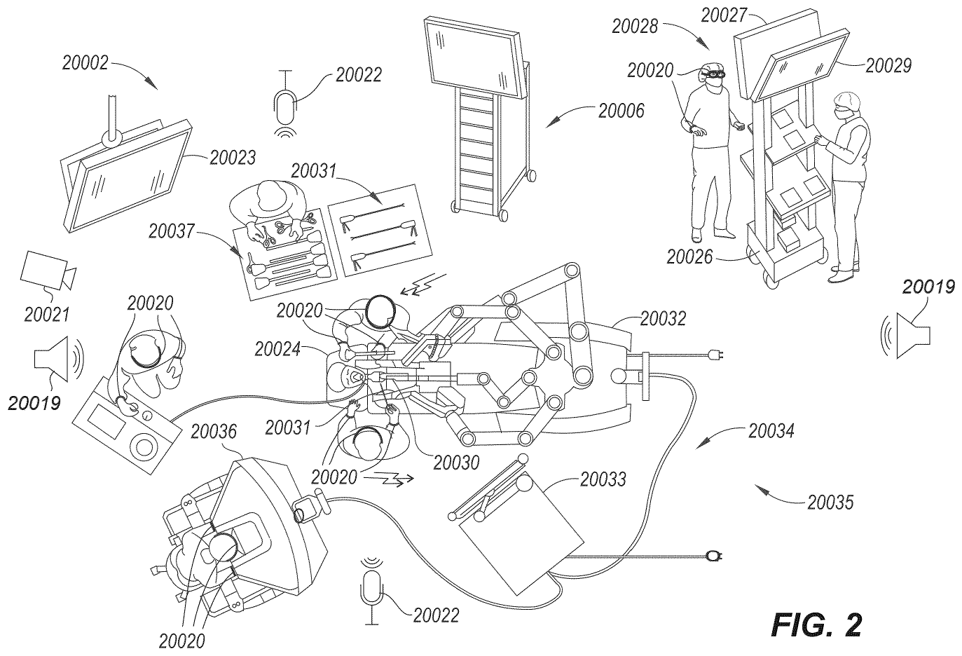

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

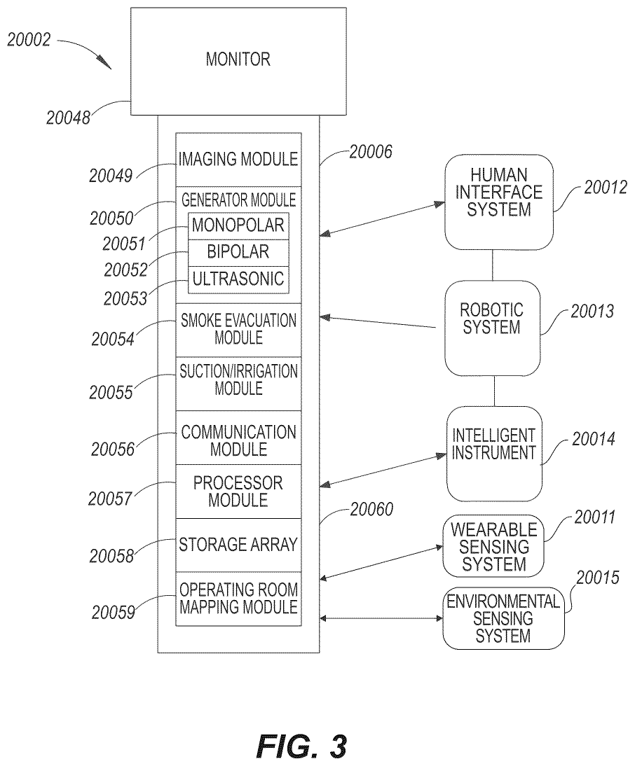

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

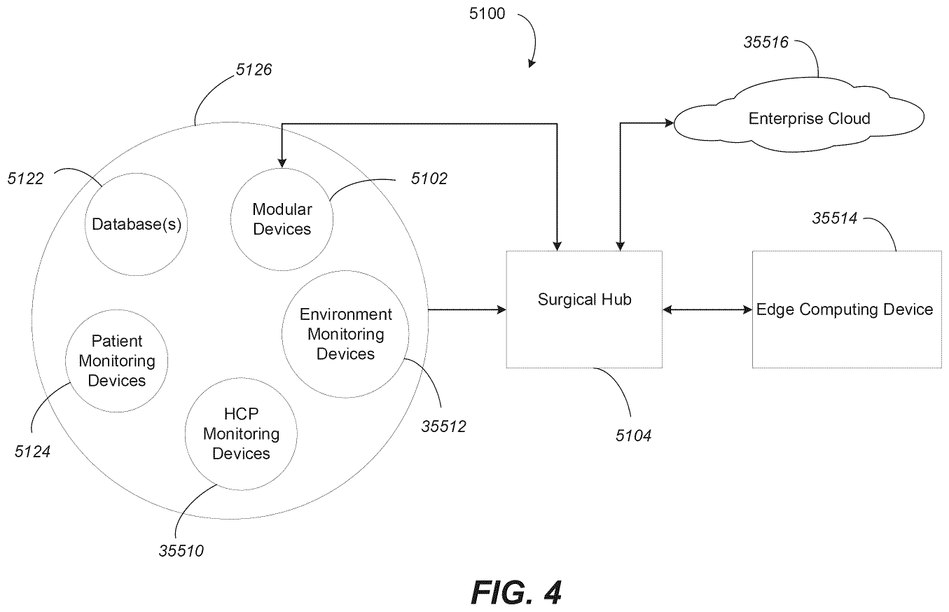

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient monitoring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

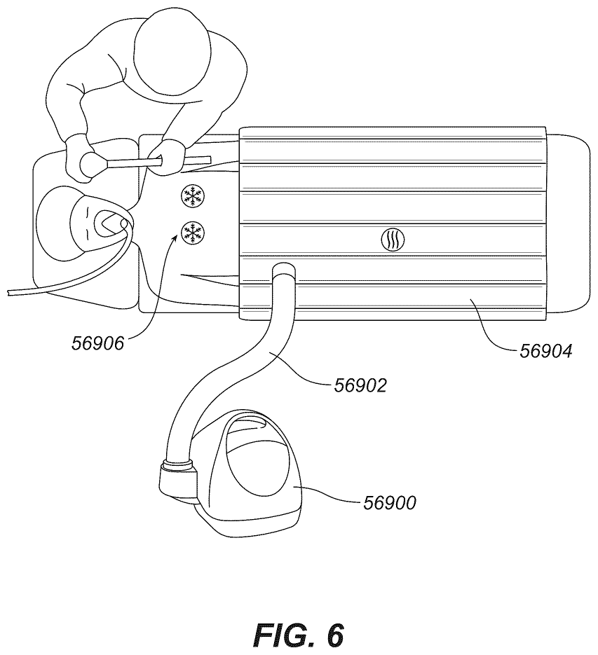

FIG. 6 illustrates a patient in an operating bed during a heart ablation procedure. The patient may be covered by a warming blanket 56904 (e.g., a bear hugger) to prevent hypothermia, as depicted on the majority of the body, with tubing 56902 connected to the warming device 56900. The bear hugger may serve to stabilize the patient's body temperature, addressing the physiologic function supplementation of thermal management.

The warming blanket 56904 may cover the patient's body while areas under the blanket appear warm, represented visually by a heat symbol (e.g., as seen in FIG. 6), reflecting the active thermal management provided during the procedure. The bear hugger system may work adjacent with the local cooling device 56906, which may introduce localized cooling to the chest area, particularly around the heart, represented by snowflake symbols indicating the cooler temperature zone. Such a configuration may be associated with a second physiologic measurement of temperature control.

The local cooling device 56906 may be positioned adjacent to the heart to protect it from overheating during the ablation procedure. The cooling system may enable tissue being targeted for ablation to not suffer collateral damage due to excessive heat from the procedure. The cooling system may be associated with managing heat generation from the ablation and maintaining a safe operating environment for the heart, aligning with feedback and adjustment mechanisms discussed herein, which may evaluate the progression of the physiologic function toward an outcome (e.g., a desired outcome).

Examples described herein may be associated with managing multiple physiologic functions simultaneously, for example, thermal regulation and cardiac cooling, in order to monitoring multiple physiologic parameters and adjusting accordingly to avoid system failure or patient instability.

The surgical device may monitor the effect of a control signal (e.g., a first/second control signal) on the physiologic function of the patient in real-time, particularly focusing on the patient's heart rate and the tissue temperature during ablation. Feedback from the local cooling device and the warming system may be continuously analyzed, so the physiologic function remains within a predefined safe range. Examples described herein may help adjust heating and cooling aspects (e.g., separately or together), which may be associated with generating control signals to adjust the physiologic function supplementation based on real-time data from one or more of the local cooling device or the warming system.

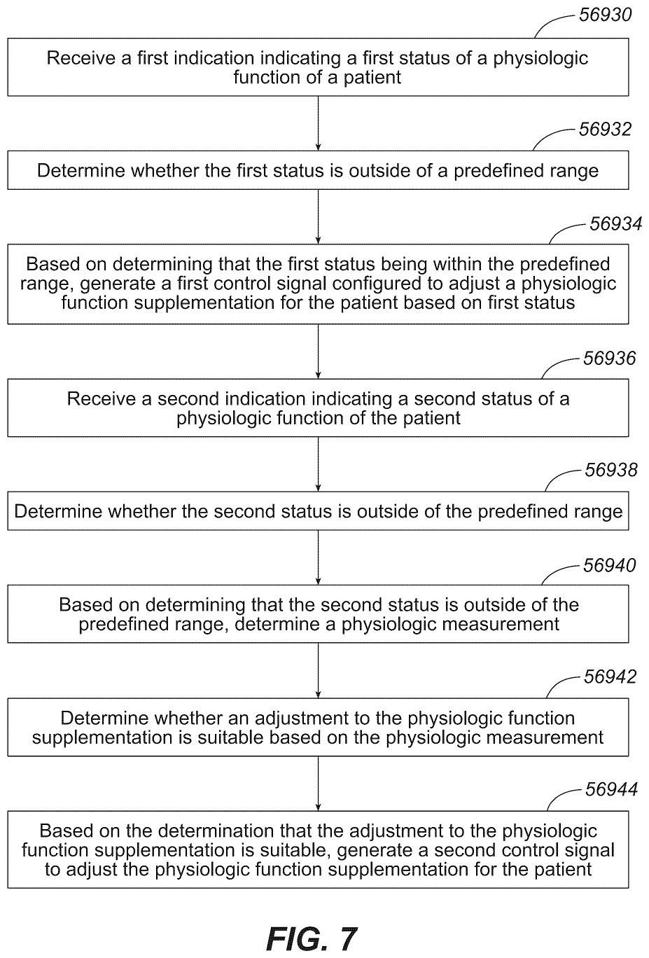

FIG. 7 illustrates a flow diagram depicting the steps involved in monitoring and adjusting physiologic function supplementation based on real-time data inputs. At 56930, a surgical device may receive a first indication indicating a first status of a physiologic function of a patient. At 56932, the device may determine whether the first status is outside of a predefined range. At 56394, the device may, based on determining that the first status being within the predefined range, generate a first control signal configured to adjust a physiologic function supplementation for the patient based on the first status. At 56936, the device may receive a second indication indicating a second status of a physiologic function of the patient. At 56938, the device may determine whether the second status is outside of the predefined range. At 56940, the device may, based on determining that the second status is outside of the predefined range, determine a physiologic measurement. At 56942, the device may determine whether an adjustment to the physiologic function supplementation is suitable based on the physiologic measurement. At 56944, the device may, based on the determination that the adjustment to the physiologic function supplementation is suitable, generate a second control signal to adjust the physiologic function supplementation for the patient.

Off-screen locations and motions of devices may be tracked or monitored. The system may track or monitor the off-screen locations and movements of surgical devices during a procedure, including the internal parameters such as temperature and activation status. The tracking may allow surgeons to have visibility and control of instruments that are not visible on-screen. By providing real-time data on device movement and status, the system may help surgical outcomes by enabling tools to operate within desired parameters and locations when the tools are outside the field of view.

Features described herein may be associated with surgical technical errors (e.g., surgeon technical errors). The system may assist surgeons in identifying off-screen tissue damage that may require repair, as well as identifying inadvertent tissue contact during laparoscopic or robotic procedures. The tracking capability may improve the speed and accuracy of intraoperative interventions by providing real-time feedback to the surgeon. By preventing or identifying technical errors, the system may affect the efficiency of the procedure and reduce the risk of unintended consequences during surgery.

Laparoscopic devices may be associated with a real-time trajectory and orientation. The system may determine the real-time trajectory and orientation of laparoscopic devices during a surgical procedure. The system may detect stationary moments, rotations, translations, and (e.g., other) motions of the device during use. The motions may include when the device is in hand, inserted, or removed. The system may utilize an inertial measurement unit (IMU) with an accelerometer and sensor fusion data, including quaternions, to calculate the position and orientation of the device's movements. When combined with a time-of-flight (ToF) sensor, the system may accurately identify the position and orientation of a trocar, which may be used in post-operative reports and affect the efficiency and precision of future procedures.

Laparoscopic Devices may be associated with a point-to-point distance measurement. The system may determine the point-to-point distance between a laparoscopic device and anatomical regions during a surgical procedure. Objectives may include quantifying the size of these regions and using the IMU's accelerometer and sensor fusion data to track the device's real-time position. The system may display the information on the surgical monitor to allow the surgical team to gain insight into measurements and locations of the device during the procedure, for example.

A real-time position of the tool relative to the abdominal wall may be established. The system may establish the real-time position of the tool relative to the patient's abdominal wall, which may serve as the fulcrum or pivot for laparoscopic surgical tools. Objectives may include measuring the distance between a known reference point on the tool and the trocar or patient. The distance may be associated with capturing and presenting the tool's movement, trajectory, and position in real-time during the surgery. The data associated with capturing and presenting the tool's movement, trajectory, and position may be included in post-operative reports to affect efficiency, focus, and procedure flow in future surgeries and, for example, affecting training and reducing variability in surgical performance.

The device may apply a maximum adjustment limit to the second control signal, such that the adjustment is associated with a safe operational limit. The device may determine a current target adjustment associated with the second control signal. The device may determine whether the current target adjustment is above the maximum adjustment limit. The device may, based on the determination that the current target adjustment is above the maximum adjustment limit, provide feedback on an effect of the second control signal.

The device may apply a maximum rate of adjustment to the second control signal, such that the adjustment is associated with a safe operational limit. The device may determine a current rate of physiologic function adjustment associated with the second control signal.

The device may determine whether the current rate of physiologic function is above the maximum rate of adjustment. The device may, based on the determination that the current rate of physiologic function is above the maximum rate of adjustment, provide feedback on an effect of the second control signal. The device may monitor an effect of the second control signal on the physiologic function of patient. The device may, based on the effect of the second control signal on the physiologic function of patient, generate a third control signal configured to adjust the physiologic function supplementation for the patient.

The device may receive a third indication indicating a third status of the physiologic function of the patient. The device may determine whether the third status is outside of the predefined range. The device may, based on determining that the third status is outside of the predefined range, determine a second physiologic measurement. The device may determine whether an adjustment to the physiologic function supplementation is suitable based on the second physiologic measurement. The device may, based on the determination that the adjustment is not suitable, generate a third control signal to pause the adjustment of the physiological function supplementation.

The device may pause the adjustment of the physiological function supplementation based on detecting an instability. The device may determine whether the physiologic function returns within the predefined range. The device may, based on the determination that the physiologic function returns within the predefined range, generate a third control signal to resume the adjustment of the physiologic function supplementation.

The device may detect that the physiologic function supplementation has attempted a predefined number of adjustments. The device may determine a failure to achieve an expected outcome based on the physiologic function supplementation having attempted the predefined number of adjustments. The device may determine that the failure to achieve the expected outcome indicates a malfunction in the physiologic function supplementation. The device may generate an alert configured to alert a user of the malfunction in the physiologic function supplementation.

The device may, based on determining that the second status is outside of the predefined range, introduce an intentional perturbation to evaluate a response of the physiologic function. The device may determine, based on the response of the physiologic function, an updated physiologic function. The device may compare the updated physiologic function against the physiologic function. The device may, based on the comparison of the updated physiologic function against the physiologic function, determine whether the physiologic function supplementation is progressing the physiologic function toward an expected outcome of the physiologic function, wherein the determination that the adjustment is suitable is further based on the physiologic function supplementation progressing the physiologic function toward the expected outcome of the physiologic function.

The device may receive the second indication from a second device as a feedback signal associated with closed-loop control of the of the device, wherein the feedback signal is associated with the adjustment of the physiologic function supplementation.

The device may adjust the physiologic function supplementation by applying a localized temperature to the patient, wherein applying the localized temperature to the patient manages a temperature of an area of a body of the patient during a medical procedure.

The device may determine that the first indication comprises a mapping indication indicating a location of abnormal tissue within the patient. The device may determine that the first control signal adjusts the physiologic function supplementation based on the location of the abnormal tissue within the patient. The device may monitor for a response of the adjusted physiologic function. The device may, based on receiving the response of the adjusted physiologic function, further adjust the physiologic function supplementation to target the abnormal tissue.

The device may determine that the first indication comprises an indication of a high-movement area within a heart of the patient. The device may determine that the first status is associated with an amount of movement of the high-movement area. The device may, based on determining that the first status is associated with the movement of the high-movement area, determine that the first control signal is associated with positioning a catheter at a site of the high-movement area comprising abnormal cells;

The device may determine that the second status is associated with a size and depth of a lesion affected by the catheter. The device may determine whether the size and depth of the lesion are sufficient to destroy the abnormal cells without excessively damaging collateral tissue, by comparing the lesion size and depth to a predefined threshold for safe tissue destruction. The device may, based on determining that the lesion size and depth are sufficient and within the predefined threshold, determine that the second control signal is generated to confirm that the physiologic function remains within a desired range post-ablation.

FIG. 7 illustrates an example machine learning diagram that may be used to process user data and health outcomes for adaptive intervention and feedback control. The system may include input layers 56960 where data sources may be integrated, such as surgical goals, surgical engagement, and physiological input data. The input data sources may include surgical demographic information, physiologic input data, and health-related inputs. An input layer may contribute to the processing of user data within the ML model, allowing the system to adapt to the individual user.

The middle section 56962 of FIG. 7 represents the processing layer of the ML model. Such layers may include tasks such as physiologic function adjustment control, real-time feedback monitoring, and adaptive control. In the processing layer, the system may perform tasks such as data normalization, perturbation assessment to evaluate physiologic responses, and signal processing for noise reduction. The system may assess whether the received data and resulting control signals are within the expected ranges, and adjust during an ongoing medical procedures, for example. Control systems may be embedded in the processing layer 56962.

Features described herein may be associated with physiologic adaptation maintenance and correctional guidance. The system for physiologic adaptation maintenance and correctional guidance may manage the adjustment of physiologic function supplementation control. The system may operate within a closed loop, where thresholds that are within an acceptable range may be adjusted. If it is detected that a physiologic indicator moves outside of a predefined range, an externally provided additional measure of the physiologic system may be used for comparative logic to determine whether adjustment is to be necessary. The comparative logic may be used such that adjustments may not be made prematurely or unnecessarily. The controlled system may limit the maximum adjustment and the rate of adjustment and may provide a feedback mechanism that monitors the current control state relative to the limit. The system may include a notification mechanism that alerts the user if a problem with the input or controlled variable is detected.

Control system logic may involve multiple types of systems, including independent control systems, nested control systems, and peer-to-peer control systems. Independent control systems may operate autonomously. Nested control systems may include subsystems functioning within a hierarchical structure. Peer-to-peer control systems may allow systems to communicate directly with one another to maintain the physiologic function of the patient. In nested control systems, a first subsystem may be paused and a second subsystem may continue to operate. The pause may allow the system to assess the effects of prior actions before implementing further adjustments. For example, a medication-induced heart rate increase may be handled by temporarily pausing adjustments to see if the physiologic parameters naturally return to baseline.

Changeover between control systems may include pausing the current control actions to allow the system to evaluate the ongoing physiologic effects of previously-made adjustments. The pause may function such that (e.g., unnecessary) overcorrection does not occur. For example, a surgical staff administering a medication may cause a temporary increase in heart rate. The system may (or may not) immediately compensate for the change, as the side effect may subside naturally. Before taking compensatory actions that may result in overcompensation, the system may pause to allow physiologic parameters to return to acceptable levels naturally. Temporary adjustments may be made based on planned interactions, such as surgical steps or the administration of additional medication.

Limits related to the control loop may be established. Limits within the control loop may be adjusted based on the patient's criticality, health condition, and procedural steps. For example, limits may change during different stages of the procedure or based on secondary conditions. The system may normalize the limits based on individual patient characteristics such as age, weight, ethnicity, and gender, using limits derived from established medical knowledge. The system may adjust limits based on the patient's pre-operative data and ongoing real-time data during the procedure. The normalization may consider factors such as the patient's reaction to anesthesia, anxiety, or other conditions, for example. The limits may be established through trending and statistical data and using vital signs to track and adjust the rate of change for control of physiologic function.

Changes in indirect monitoring metrics of the control system may be made prior to external adjustment to evaluate patient response. The system may handle variable external biomarkers within the closed loop of smart system control to manage changes in indirect monitoring metrics. The control system may adjust based on these biomarkers before making external adjustments. Indirect monitoring may allow the system to assess physiologic variables that are not directly under its control. The system may rely on external inputs, like secondary biomarkers, to determine whether an adjustment to physiologic supplementation is necessary.

The control system may handle physiologic components. The control system may manage physiologic components directly by controlling various physiologic functions through control signals. When the control signals reach their response limits, secondary actions may be taken on those control signals to determine the validity of the response. For example, if a control signal has attempted to adjust blood pressure or heart rate, and the response falls outside of the expected range, the system may perform actions to verify whether the physiologic response is genuine or the result of noise or sensor error.

Adjustments may be based on response validation. For example, the system may monitor blood pressure, heart rate, and other patient vitals consistently, and if the system detects a spike in response, the system may determine whether the spike is real or due to external interference. The system may increase the sampling rate (e.g., drastically) to analyze the validity of the signal and to determine if the signal represents noise or is a genuine response. If the system determines that the spike falls outside its capabilities to control, the system may attempt secondary actions such as adjusting how the signal is processed, before making further interventions.

In examples, a sampling rate may be modified and filtered. To address undersampling or oversampling, the system may modify the sampling rate to avoid aliasing (e.g., for accurate signal interpretation). If signals appear to be undersampled, data points (e.g., additional data points) may be gathered to provide an understanding of the physiologic trends. In oversampling, the system may introduce signal filtering, such as a low pass filter, to manage the incoming data. Hysteresis may be applied to gauge signal responses, so that fluctuations are accounted for before adjustments to the physiologic supplementation are made. Such may reduce the risk of unnecessary interventions due to temporary anomalies in the patient's physiologic readings.

Sample filtering and/or control may be included in examples described herein. The system may introduce signal processing or filtering to incoming signals to improve data accuracy before making adjustments to physiologic function. For example, a low pass filter may be used to remove high-frequency noise from the signals. By applying the filter, the system may determine that data used to adjust physiologic functions, such as heart rate or blood pressure, is free of erratic or spurious fluctuations that could lead to unnecessary or inaccurate interventions. The filtering may improve the precision of the control system in managing physiologic supplementation.

Hysteresis may occur on the signal to gauge a response. The system may use hysteresis when setting limits, such that responses it receives are valid and to avoid rapid fluctuations around a boundary condition. As the system approaches a threshold, hysteresis may be applied to smoothen transition and prevent the system from making frequent adjustments as it passes over the boundary condition. The system may reduce the risk of overcompensating for minor or temporary deviations in physiologic parameters, such that responses remain consistent and stable over time.

The output section of FIG. 7 may include intervention layers 56964, where a layer of the intervention layer 56964 is configured to handle types of feedback-based intervention. The interventions may include adjustments to physiologic function based on real-time feedback, including health correction and physiologic response interventions. The system may determine a suitable intervention based on the processed input data, such that adjustments to the patient's physiologic function supplementation are tailored to their needs and are responsive to their health.

Physiologic adjustment latency may be compensated to measured response. In examples, the patient's physiologic response may lag behind the action taken by the system, e.g., adjustments may take longer to manifest in the patient's body. The system may compensate for the latency to avoid overcorrecting based on delayed feedback. For example, if a patient's heart rate takes time to respond to medication or an intervention, the system may account for the time by adjusting the rate at which it expects a corresponding response. The system may not overreact to perceived issues.

An intentional perturbation may be introduced to evaluate the physiologic response. The system may introduce an intentional perturbation, or a controlled change (e.g., a small, controlled change), to assess how the patient's physiologic system responds. This perturbation may allow the system to determine whether the patient's condition is real and in need of further intervention. For example, if the patient's temperature appears to be dropping unexpectedly, the system may briefly increase the warming of the patient (e.g., through the bear hugger) to observe whether the patient's temperature responds as expected. If the response tracks with the perturbation, the system may confirm that the patient's condition is genuine. If the response does not track with the perturbation, the system may indicate a potential equipment or control system error, prompting further diagnostic evaluation. The system may evaluate responses in locations within the body to compare relative physiologic changes across different areas.

A first smart system may issue input from a second smart system as a feedback signal for closed loop control. A first smart system may receive input from a second smart system in the form of a feedback signal to operate within a closed loop control framework. The first smart system may have situational awareness of the patient, the procedure's progress, and the surgical tasks involved, enabling the first smart system to evaluate perturbances in the control signal that may fall outside of a pre-defined window of adjustability. The evaluation may take place before the system makes more drastic adjustments. Discernment between real control variable changes and those caused by physiological impacts may allow the system to avoid unnecessary or inappropriate responses, such that adjustments occur when conducive for the patient's condition.

Perturbances may arise from changes in the measurement system, such as when a sensor may fall out of patient contact, or if signal interference may occur. In examples, the system may seek a secondary external source of data to confirm whether the perturbance is real or if it is caused by a known physiologic interaction. If the perturbance is determined to be caused by the physiologic system or a known external factor, the system may choose to ignore the perturbance or compensate for the perturbance while allowing the closed-loop control system to continue operating as normal. The ability to differentiate between equipment-related anomalies and true physiologic changes may enable the system to maintain accuracy in its feedback control process.

Features described herein may include adaptive physiologic thermal management. The system may be capable of adaptive physiologic thermal management, such as localized organ cooling for protective temperature control. In procedures such as catheter ablation for atrial fibrillation (AFib), synchronization and precise positioning of the radiofrequency (RF) ablation application may be associated with treatment (e.g., effective treatment). While the heart may continue beating, the system may use the smart control loop for accurate localization of the treatment site, preventing damage to surrounding heart tissue. The first smart system may receive feedback about the ablation site from the second smart system to track the progress of the procedure.

Features described herein may be associated with catheter ablation of the heart for atrial fibrillation (AFib). For procedures like AFib, the healthcare provider (HCP) may use an electrode catheter to send or measure small electrical impulses to map the heart and locate abnormal tissues responsible for arrhythmia. The mapping process may be performed during stable sinus rhythm (SR), where at least three characteristics—such as fluoroscopic position, catheter drop, atrial potential, or impedance decrease—may be used to determine the heart's left atrial-pulmonary vein junction. Once the abnormal tissue is located, RF energy may be applied to a small, targeted area of heart tissue to destroy cells causing rapid heartbeats. The lesion's size may be controlled through a balance of heat conduction from the RF electrode and heat loss to the blood pool. The system may use feedback to determine the appropriate power and duration of ablation while avoiding excessive damage to surrounding tissues.

Sweeping ablation may be conducted for heart conduction remodeling. Sweeping ablation, which involves remodeling the heart's conduction system over a wider area, may be associated with the system moving the ablation tip over a predetermined region. Such may address irregular or uncoordinated contractions of the heart muscle. The system may control the force and location of the ablation tip dynamically, enabling a (e.g., proper) time-on-target, power levels, and the desired depth of tissue penetration. The combination of factors may allow for the effective treatment of arrhythmias without causing damage to collateral heart muscle and structures.

Heart electrical system mapping may be associated with ablation tracking on a map. A mapping module (e.g., a Carto Prime Mapping Module from Biosense Webster) may be utilized to integrate multiple heart electrical pathways and activations to create a visualization of arrhythmia origins and their interactions. The mapping system may provide capabilities for identifying and tracking electrical disruptions within the heart. Coherent mapping may be employed to simplify the diagnosis of scar-related complex atrial arrhythmias by applying physiological constraints on local activation times (LAT) information. Such coherent mapping may reduce time associated with arrhythmia termination compared to ablation guided by mapping algorithms. Through visualization capabilities, the mapping module (e.g., Carto Prime Module) may allow healthcare providers to target the ablation areas for treatment.

The system's mapping capabilities may be associated with identifying irregular atrial arrhythmias, such as irregular atrial arrythmias with repetitive focal and rotational activations. With parallel mapping, the system may map (e.g., simultaneously map) arrhythmias (e.g., different arrythmias) using the same catheter locations, allowing for (e.g., comprehensive) treatment planning. LAT Hybrid may increase location accuracy by associating LAT information for premature ventricular complexes (PVC) with its corresponding normal sinus rhythm location. The use of Octaray mapping catheters from Biosense Webster may be associated with a high (e.g., higher) resolution and detail of the electrical mappings, allowing for (e.g., precise) targeting of abnormal areas within the heart.

Pulsed field ablation (PFA) may be described herein and may use electrical field application. PFA, e.g., electroporation, may cause temporary pore-like openings in cell walls, which may promote cell death and lead to an ablation of the target tissue. PFA may provide advantages over thermal ablation, for example, by reducing the risk of collateral damage and offering more controlled lesion creation. The technology may be associated with reducing complications associated with overheating or damage to adjacent tissues during heart ablation procedures.

Features described herein may be associated with electrode-tissue interface and cooling management. The size of the distal electrode and the application of saline cooling may be associated with minimizing impedance rises and improving lesion control during ablation. By controlling the pressure applied by the electrode, as well as the power level, the system may create lesions (e.g., large/deep lesions) with sufficient control. The electrode-tissue interface may reach a temperature of 50° C. or higher to cause tissue necrosis, and care may be taken to prevent the temperature from reaching 100° C., which may result in coagulation of denatured proteins and plasma on the catheter tip. This coagulation may impede the current delivery and increase the risk of thromboembolic complications. Control of saline deposition, temperature, and volume may be associated with achieving tissue effects without introducing additional risks.

Features described herein may be associated with saline management and power control during atrial fibrillation (AF) ablation. During AF ablation in stable sinus rhythm (SR), RF pulses may be delivered with temperature settings (e.g., up to 55° C.) and energy levels (e.g., up to 50 W) for the 8-mm-tip ablation catheter, or temperature settings (e.g., 43° C.) and energy levels (e.g., 35 W) for the irrigated-tip catheter. The settings may be maintained until local electrogram amplitude is reduced by at least 80% or decreased below 0.1 mV for a duration (e.g., up to 120 seconds). The system may be associated with managing the deposition rate, temperature, and volume of saline to control the local cooling effects at the ablation site. The interconnected control of power levels, saline heat mitigation, electrode contact zone, and pressure (e.g., energy density) may allow for balancing cauterization magnitude, lesion depth, and avoidance of collateral damage during ablation procedures. This may be associated with the system's ability to treat the Afib while minimizing harm to surrounding heart tissue and maintaining effective control over the ablation process.

Features describe herein may be associated with atrial fibrillation (Afib). Afib may be associated with an irregular heart rhythm that originates in the upper chambers of the heart (e.g., the atria). Afib may result from fast and irregular beats within the atria, which may reach more than 400 beats per minute. In a healthy heart, the muscle may contract regularly, allowing for efficient blood flow. During Afib, the irregular contractions may disrupt such efficient blood flow.

Persistent Afib may be a condition that lasts for more than one week and may require treatment to restore the heart's rhythm. Long-standing persistent Afib, which may persist for over a year, may be more difficult to manage and may be associated with additional intervention. The forms of Afib may be associated with challenges in treatment due to the prolonged nature of the irregular heart rhythm.

The symptoms of Afib may be associated with extreme fatigue, irregular heartbeat, and heart palpitations. Patients may describe the palpitations as feeling like butterflies or a fish flopping in their chest. Symptoms may be associated with dizziness or lightheadedness, fainting (e.g., syncope), shortness of breath (e.g., dyspnea), and chest pain (e.g., angina). These symptoms may vary in severity depending on the patient's condition.

Features described herein may be associated with the clinical manifestation of Afib. Afib may be associated with symptoms that are often uncomfortable and disruptive to the patient. The irregular heart rhythm may be associated with sensations of fatigue and palpitations, and symptoms (e.g., shortness of breath and chest pain) may complicate the condition. Depending on the duration and severity of the Afib episode, the symptoms may be associated with medical intervention.

Features described herein may be associated with the management of perioperative hypothermia. Perioperative hypothermia may occur inadvertently during anesthesia, aided ventilation, and surgical operating room (OR) losses. Hypothermia may be a monitored complication during anesthesia and surgery. Hypothermia may result in cardiac abnormalities, impaired wound healing, increased surgical site infections, shivering, delayed postoperative recovery, and coagulopathies. Hypothermia may be an undesirable event in elective surgery. 70-90% of patients may experience hypothermia within an (e.g., one) hour after the initiation of surgery.

Features described herein may be associated with perioperative hypothermia management. Active temperature management protocols may (or may not) be widely implemented, due to, for example, misconceptions (e.g., that forced-air warming increases infection rates), surgeons' complaints of discomfort, inconsistent temperature monitoring, and a lack of appreciation of the causes and consequences of perioperative hypothermia.

Perioperative hypothermia may be addressed. The National Institute for Clinical Excellence (NICE) has published guidelines for the management of inadvertent perioperative hypothermia in adults. Such guidelines may include forced-air warming to be initiated as early as possible, in the anesthetic room, for surgeries lasting longer than 30 minutes or for patients with two or more risk factors for hypothermia. Intravenous fluids greater than 500 ml may be warmed. Intraoperative core temperature monitoring may conduct every 30 minutes for thermal management and to prevent both hypothermia and overheating.

Features described herein may be associated with postoperative considerations for hypothermia. After surgery, patients may, for example, not be discharged from recovery areas until their temperature reaches 36.0° C. Temperature monitoring may continue with the same frequency as postoperative observations for the first 24 hours in inpatients. For outpatient surgeries, normothermia may be a prerequisite for discharge. Deliberate intraoperative hypothermia may be employed as a protective strategy for vital organs during procedures, such as cardiac surgery, where deep hypothermia (below 28° C.) may enable safe circulatory arrest.

1. A surgical device, the surgical device comprising:
a processor configured to:
receive a first indication indicating a first status of a physiologic function of a patient;
determine whether the first status is outside of a predefined range;
based on determining that the first status being within the predefined range, generate a first control signal to configured to adjust a physiologic function supplementation for the patient based on the first status;
receive a second indication indicating a second status of a physiologic function of the patient;
determine whether the second status is outside of the predefined range;
based on determining that the second status is outside of the predefined range, determine a physiologic measurement;
determine whether an adjustment to the physiologic function supplementation is suitable based on the physiologic measurement; and
based on the determination that the adjustment to the physiologic function supplementation is suitable, generate a second control signal to adjust the physiologic function supplementation for the patient.

2. The device of claim 1, wherein the processor is further configured to:
apply a maximum adjustment limit to the second control signal, such that the adjustment is associated with a safe operational limit;
determine a current target adjustment associated with the second control signal;
determine whether the current target adjustment is above the maximum adjustment limit; and
based on the determination that the current target adjustment is above the maximum adjustment limit, provide feedback on an effect of the second control signal.

3. The device of claim 1, wherein the processor is further configured to:
apply a maximum rate of adjustment to the second control signal, such that the adjustment is associated with a safe operational limit;
determine a current rate of physiologic function adjustment associated with the second control signal; and
determine whether the current rate of physiologic function is above the maximum rate of adjustment; and
based on the determination that the current rate of physiologic function is above the maximum rate of adjustment, provide feedback on an effect of the second control signal.

4. The device of claim 1, wherein the device is further configured to:
monitor an effect of the second control signal on the physiologic function of patient;
based on the effect of the second control signal on the physiologic function of patient, generate a third control signal configured to adjust the physiologic function supplementation for the patient.

5. The device of claim 1, wherein the processor is further configured to:
pause the adjustment of the physiological function supplementation based on detecting an instability;
determine whether the physiologic function returns within the predefined range; and
based on the determination that the physiologic function returns within the predefined range, generate a third control signal to resume the adjustment of the physiologic function supplementation.

6. The device of claim 1, wherein the processor is further configured to:
detect that the physiologic function supplementation has attempted a predefined number of adjustments;
determine a failure to achieve an expected outcome based on the physiologic function supplementation having attempted the predefined number of adjustments;
determine that the failure to achieve the expected outcome indicates a malfunction in the physiologic function supplementation; and
generate an alert configured to alert a user of the malfunction in the physiologic function supplementation.

7. The device of claim 1, wherein the processor is further configured to:
based on determining that the second status is outside of the predefined range, introduce an intentional perturbation to evaluate a response of the physiologic function;
determine, based on the response of the physiologic function, an updated physiologic function;
compare the updated physiologic function against the physiologic function; and
based on the comparison of the updated physiologic function against the physiologic function, determine whether the physiologic function supplementation is progressing the physiologic function toward an expected outcome of the physiologic function, wherein the determination that the adjustment is suitable is further based on the physiologic function supplementation progressing the physiologic function toward the expected outcome of the physiologic function.

8. The device of claim 1, wherein the processor is further configured to:
determine that the first indication comprises an indication of a high-movement area within a heart of the patient;
determine that the first status is associated with an amount of movement of the high-movement area;
based on determining that the first status is associated with the movement of the high-movement area, determine that the first control signal is associated with positioning a catheter at a site of the high-movement area comprising abnormal cells;
determine that the second status is associated with a size and depth of a lesion affected by the catheter;
determine whether the size and depth of the lesion are sufficient to destroy the abnormal cells without excessively damaging collateral tissue, by comparing the lesion size and depth to a predefined threshold for safe tissue destruction;
based on determining that the lesion size and depth are sufficient and within the predefined threshold, determine that the second control signal is generated to confirm that the physiologic function remains within a desired range post-ablation.

9. A method for a surgical device, the method comprising:
receiving a first indication indicating a first status of a physiologic function of a patient;
determining whether the first status is outside of a predefined range;
based on determining that the first status being within the predefined range, generating a first control signal to configured to adjust a physiologic function supplementation for the patient based on the first status;
receiving a second indication indicating a second status of a physiologic function of the patient;
determining whether the second status is outside of the predefined range;
based on determining that the second status is outside of the predefined range, determining a physiologic measurement;
determining whether an adjustment to the physiologic function supplementation is suitable based on the physiologic measurement; and
based on determining that the adjustment to the physiologic function supplementation is suitable, generating a second control signal to adjust the physiologic function supplementation for the patient.

10. The method of claim 9, wherein the method further comprises:
applying a maximum adjustment limit to the second control signal, such that the adjustment is associated with a safe operational limit;
determining a current target adjustment associated with the second control signal;
determining whether the current target adjustment is above the maximum adjustment limit; and
based on determining that the current target adjustment is above the maximum adjustment limit, providing feedback on an effect of the second control signal.

11. The method of claim 9, wherein the method further comprises:
applying a maximum rate of adjustment to the second control signal, such that the adjustment is associated with a safe operational limit;
determining a current rate of physiologic function adjustment associated with the second control signal; and
determining whether the current rate of physiologic function is above the maximum rate of adjustment; and
based on the determination that the current rate of physiologic function is above the maximum rate of adjustment, providing feedback on an effect of the second control signal.

12. The method of claim 9, wherein the method further comprises:
monitoring an effect of the second control signal on the physiologic function of patient;
based on the effect of the second control signal on the physiologic function of patient, generating a third control signal configured to adjust the physiologic function supplementation for the patient.

13. The method of claim 9, wherein the method further comprises:
pausing the adjustment of the physiological function supplementation based on detecting an instability;
determining whether the physiologic function returns within the predefined range; and
based on determining that the physiologic function returns within the predefined range, generate a third control signal to resume the adjustment of the physiologic function supplementation.

14. The method of claim 9, wherein the method further comprises:
detecting that the physiologic function supplementation has attempted a predefined number of adjustments;
determining a failure to achieve an expected outcome based on the physiologic function supplementation having attempted the predefined number of adjustments;
determining that the failure to achieve the expected outcome indicates a malfunction in the physiologic function supplementation; and
generating an alert configured to alert a user of the malfunction in the physiologic function supplementation.

15. The method of claim 9, wherein the method further comprises:
based on determining that the second status is outside of the predefined range, introducing an intentional perturbation to evaluate a response of the physiologic function;
determining, based on the response of the physiologic function, an updated physiologic function;
comparing the updated physiologic function against the physiologic function; and
based on the comparison of the updated physiologic function against the physiologic function, determining whether the physiologic function supplementation is progressing the physiologic function toward an expected outcome of the physiologic function, wherein determining that the adjustment is suitable is further based on the physiologic function supplementation progressing the physiologic function toward the expected outcome of the physiologic function.

16. The method of claim 9, wherein the method further comprises:
determining that the first indication comprises an indication of a high-movement area within a heart of the patient;
determining that the first status is associated with an amount of movement of the high-movement area;
based on determining that the first status is associated with the movement of the high-movement area, determining that the first control signal is associated with positioning a catheter at a site of the high-movement area comprising abnormal cells;
determining that the second status is associated with a size and depth of a lesion affected by the catheter;
determining whether the size and depth of the lesion are sufficient to destroy the abnormal cells without excessively damaging collateral tissue, by comparing the lesion size and depth to a predefined threshold for safe tissue destruction;
based on determining that the lesion size and depth are sufficient and within the predefined threshold, determining that the second control signal is generated to confirm that the physiologic function remains within a desired range post-ablation.

17. A surgical device, the surgical device comprising:
a processor configured to:
receive a first indication indicating a first status of a physiologic function of a patient;
determine whether the first status is outside of a predefined range;
based on determining that the first status being within the predefined range, generate a first control signal to configured to adjust a physiologic function supplementation for the patient based on the first status;
receive a second indication indicating a second status of a physiologic function of the patient;
determine a physiologic measurement;
determine whether an adjustment to the physiologic function supplementation is suitable based on the physiologic measurement; and
based on the determination that the adjustment to the physiologic function supplementation is suitable, generate a second control signal to adjust the physiologic function supplementation for the patient.

18. The device of claim 17, wherein the processor is further configured to:
apply a maximum adjustment limit to the second control signal, such that the adjustment is associated with a safe operational limit;
determine a current target adjustment associated with the second control signal;
determine whether the current target adjustment is above the maximum adjustment limit; and
based on the determination that the current target adjustment is above the maximum adjustment limit, provide feedback on an effect of the second control signal.

19. The device of claim 17, wherein the processor is further configured to:
apply a maximum rate of adjustment to the second control signal, such that the adjustment is associated with a safe operational limit;
determine a current rate of physiologic function adjustment associated with the second control signal; and
determine whether the current rate of physiologic function is above the maximum rate of adjustment; and
based on the determination that the current rate of physiologic function is above the maximum rate of adjustment, provide feedback on an effect of the second control signal.

20. The device of claim 17, wherein the device is further configured to:
monitor an effect of the second control signal on the physiologic function of patient;
based on the effect of the second control signal on the physiologic function of patient, generate a third control signal configured to adjust the physiologic function supplementation for the patient.
